(19) United States
(12) Patent Application Publication
(10) Pub. No.: US 2021/0186305 A1
(43) Pub. Date: Jun. 24, 2021

(54) DEFLECTABLE MEDICAL PROBE HAVING IMPROVED RESISTANCE TO FORCES APPLIED IN ROTATION

(71) Applicant: BIOSENSE WEBSTER (ISRAEL) LTD., Yokneam (IL)

(72) Inventors: Yehuda Algawi, Binyamina (IL); Assaf Govari, Haifa (IL); Ilya Sitnitsky, Nahariya (IL); Gili Attias, Haifa (IL)

(21) Appl. No.: 16/725,055

(22) Filed: Dec. 23, 2019

(51) Int. Cl. A61B 1/005; A61B 1/233

(52) U.S. Cl. CPC A61B 1/0056; A61B 1/0052; A61B 1/233

(57) ABSTRACT

A medical probe includes a shaft, for insertion into a cavity of a patient body, and a distal-end assembly. The distal-end assembly is coupled to a distal end of the shaft and including a hollow tube, which is configured to deflect relative to a longitudinal axis of the hollow tube and to rotate about the longitudinal axis. The hollow tube having (i) an intrusion, having at least a first surface, and (ii) a protrusion, which is facing the intrusion and having a second surface. When the hollow tube is deflected, at least part of the protrusion protrudes into the intrusion so that a first section of the first surface and a second section of the second surface are facing one another, and when the hollow tube is deflected and rotated, the first and second sections of the first and second surfaces apply force to one another and thus resist rotation of the hollow tube.

### FIELD OF THE INVENTION

The present invention relates generally to minimally invasive medical devices, and particularly to techniques for medical probes having improved maneuverability and durability.

### BACKGROUND OF THE INVENTION

Various types of medical probes have mechanical designs intended to improve probe durability when maneuvered in a patient body.

For example, U.S. Patent Application Publication 2011/0152880 describes an instrument for performing minimally invasive surgical procedures. The instrument includes an elongate body and a support member disposed within or along the elongate body. The support member is configured to support steering, articulation, and angular rotational movement of the elongate body, provide torsion control, and support precise and accurate placement of the distal portion of the elongate body so that complex surgical procedure may be performed using the instrument.

U.S. Patent Application Publication 2017/0325841 describes an apparatus including a tube, shaped to define a tube lumen and a distal portion that has a plurality of articulated sections. The apparatus further includes a ribbon that passes longitudinally through the tube lumen and is connected to a distalmost one of the articulated sections, and a control handle disposed at a proximal end of the tube, the control handle being configured to flex the distal portion of the tube by pulling the ribbon.

### SUMMARY OF THE INVENTION

An embodiment of the present invention that is described herein provides a medical probe, including a shaft, for insertion into a cavity of a patient body, and a distal-end assembly. The distal-end assembly is coupled to a distal end of the shaft and including a hollow tube, which is configured to deflect relative to a longitudinal axis of the hollow tube and to rotate about the longitudinal axis. The hollow tube having (i) an intrusion, having at least a first surface, and (ii) a protrusion, which is facing the intrusion and having a second surface. When the hollow tube is deflected, at least part of the protrusion protrudes into the intrusion so that a first section of the first surface and a second section of the second surface are facing one another, and when the hollow tube is deflected and rotated, the first and second sections of the first and second surfaces apply force to one another and thus resist rotation of the hollow tube.

In some embodiments, the distal-end assembly includes (i) a first slot, located at a first section along the longitudinal axis of the hollow tube, and having a first size that limits bending of the first section by a first local radius of curvature (LROC), and (ii) a second slot, located at a second different section along the longitudinal axis of the hollow tube, and having a second different size that limits bending of the second section by a second different LROC. In other embodiments, at least the first slot includes (i) a plurality of the intrusions having respective one or more first surfaces, and (ii) a plurality of the protrusions having respective one or more second surfaces, and the intrusions and protrusions are arranged along at least the first slot. In yet other embodiments, at least the first slot includes at least a given intrusion having a first given surface, and a given protrusion, which is facing the given intrusion and having a second given surface, when the hollow tube is not deflected, the first and second given surfaces do not apply force to one another.

In an embodiment, the medical probe includes a control handle, fitted at a proximal end of the shaft and configured to bend the first section by up to the first LROC and the second section by up to the second LROC. In another embodiment, the distal-end assembly includes an alloy of nickel and titanium. In yet another embodiment, the intrusion is shaped to fit over the protrusion, such that, when the hollow tube is deflected and rotated, the first and second sections do not slide relative to one another.

In some embodiments, the first and second sections of the first and second surfaces press against one another. In other embodiments, the protrusion has a shape selected from a list of shapes consisting of: rectangular, parallelogram, trapezoid, dome, and pyramid. In yet other embodiments, the cavity includes an ear-nose-throat (ENT) sinus.

There is additionally provided, in accordance with an embodiment of the present invention, a method for producing a medical probe, the method including providing a shaft for insertion into a cavity of a patient body. A distal-end assembly that includes a hollow tube that deflects relative to a longitudinal axis of the hollow tube and rotates about the longitudinal axis is coupled to a distal end of the shaft. The hollow tube has (i) an intrusion, having at least a first surface, and (ii) a protrusion, which is facing the intrusion and having a second surface. When the hollow tube is deflected, at least part of the protrusion protrudes into the intrusion so that a first section of the first surface and a second section of the second surface are facing one another, and when the hollow tube is deflected and rotated, the first and second sections of the first and second surfaces apply force to one another and thus resist rotation of the hollow tube.

In some embodiments, the method includes forming at least one of the first and second slot using a laser cutting technique.

The present invention will be more fully understood from the following detailed description of the embodiments thereof, taken together with the drawings in which:

### BRIEF DESCRIPTION OF THE DRAWINGS

FIG. 1 is a schematic, pictorial illustration of an ear-nose-throat (ENT) procedure using an ENT system, in accordance with an embodiment of the present invention; and

FIGS. 2A and 2B are schematic, pictorial illustrations of a medical probe having a deflectable and rotatable distal-end assembly, in accordance with embodiments of the present invention.

### DETAILED DESCRIPTION OF EMBODIMENTS

### Overview

Some medical procedures require insertion of a medical probe into a branched organ of a patient, such as a sinus of a patient ear-nose-throat (ENT) system. Maneuvering of the probe within the sinus to a desired location, may result in a breakage of the probe due external forces applied to the probe, e.g., by a bone of the ENT system.

Embodiments of the present invention that are described hereinbelow provide a medical probe having improved resistance to probe breakage when maneuvering the medical probe within branched organs in a patient body.

In some embodiments, the medical probe comprises a shaft for insertion into a cavity of a patient body, such as a sinus of a patient ENT system, and the distal-end assembly is coupled to the distal end of the shaft. The distal-end assembly comprises a hollow tube, which is configured to deflect relative to a longitudinal axis of the hollow tube and to rotate about the longitudinal axis.

In some embodiments, the hollow tube has at least an intrusion having at least one surface, referred to herein as a first surface. The hollow tube also has at least a protrusion, which is facing the intrusion and having at least another surface, referred to herein as a second surface.

In some embodiments, when the tube is deflected, at least part of the protrusion protrudes into the intrusion so that a first section of the first surface and a second section of the second surface are facing one another. When the hollow tube is deflected and rotated, the first and second surfaces that are facing one another, are applying force to one another, and thus, resist rotation and breakage of the hollow tube. In some embodiments, the hollow tube may comprise multiple protrusions and respective intrusions patterned along the circumference of the hollow tube, so as to improve the resistance to rotation and breakage of the hollow tube.

In some embodiments, the plurality of protrusions and intrusions may improve the deflecting ability, and therefore the flexibility, of the hollow tube. The maximal deflecting ability at a given location along the hollow tube is specified by a local radius of curvature (LROC) at that location.

In some embodiments, a first set of one or more protrusions and respective intrusions, which is located at a first, distalmost, section of the hollow tube, has a given size that limits the bending ability of the distalmost section by a predefined LROC. In some embodiments, a second set of one or more protrusions and respective intrusions, which is located along the hollow tube at a second section, proximal to the first section, has a size smaller than the given size, resulting in a LROC larger than the predefined LROC of the distalmost section.

In some embodiments, the hollow tube may comprise multiple sets of one or more protrusions and respective intrusions that are formed along the longitudinal axis of the tube, wherein the size of the protrusions and respective intrusions increases with the proximity to the distal end of the tube. Thus, the LROC corresponding to the protrusions and respective intrusions decreases with their proximity to the distal end of the tube.

In some embodiments, the distal-end assembly comprises a control handle fitted at the proximal end of the shaft. The distal-end assembly further comprises one or more pulling wires, which run through a longitudinal lumen of the tube, and are coupled to the distalmost section of the hollow tube and to the control handle. In some embodiments, a user of the medical probe may apply the pulling wires for deflecting one or more sections of the distal-end assembly up to the desired respective LROCs. Moreover, when the hollow tube is deflected the user may rotate the medical probe about the longitudinal axis without a concern of breaking within the patient ENT system.

The disclosed techniques improve the flexibility and durability of a narrow medical probe, so as to improve the maneuverability of the medical probe in rigid branched organs.

### System Description

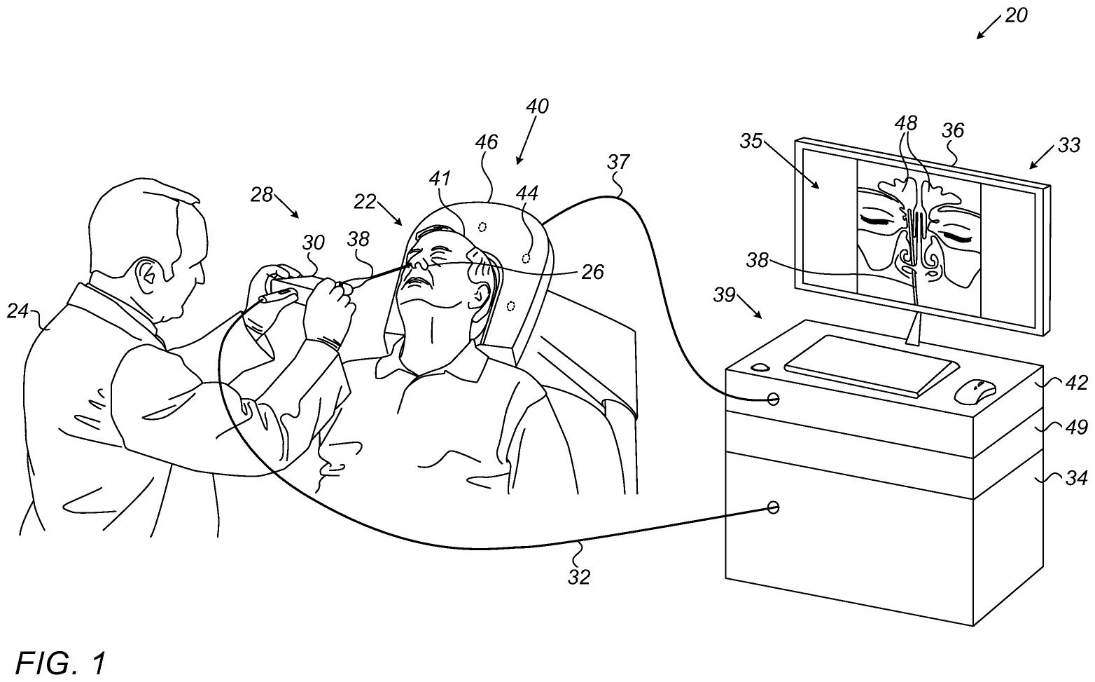

FIG. 1 is a schematic, pictorial illustration of an ear-nose-throat (ENT) procedure using an ENT system 20, in accordance with an embodiment of the present invention. In some embodiments, ENT system 20 comprises a medical probe, referred to herein as an ENT tool 28, which is configured to carry out the ENT procedure, such as but not limited to treating infection from one or more sinuses 48 of a patient 22.

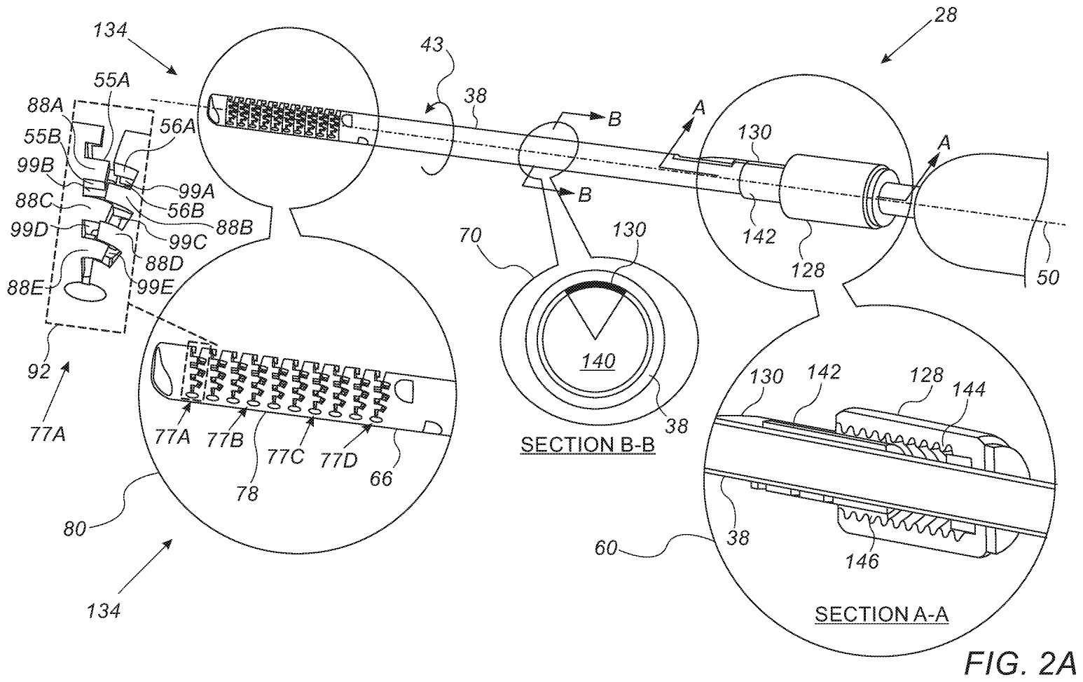

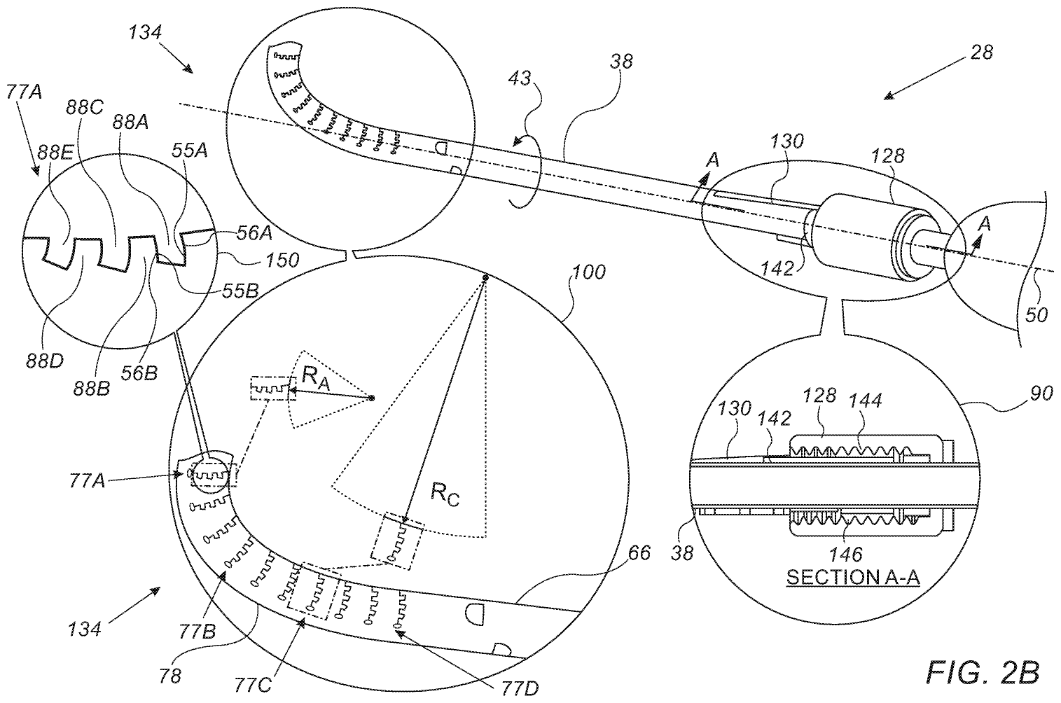

In some embodiments, ENT tool 28 comprises a shaft 38, coupled to the distal end, which a physician 24 inserts into a nose 26 of patient 22. ENT tool 28 further comprises a handheld apparatus 30, coupled to a proximal end of shaft 38 and configured to assist physician 24 in maneuvering the distal end of shaft 38 in a head 41 of patient 22. Shaft 38 is shown in detail in FIGS. 2A and 2B below.

In an embodiment, system 20 further comprises a magnetic position tracking system, which is configured to track the position of one or more position sensors in head 41. The magnetic position tracking system comprises magnetic field-generators 44 and multiple position sensors (not shown). The position sensors generate position signals in response to sensing external magnetic fields generated by field-generators 44, thereby enabling a processor 34 (described in detail below) to estimate the position of each sensor within head 41 of patient 22.

This method of position sensing is implemented in various medical applications, for example, in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1, whose disclosures are all incorporated herein by reference.

System 20 further comprises a location pad 40, which comprises field-generators 44 fixed on a frame 46. In the exemplary configuration shown in FIG. 1, pad 40 comprises five field-generators 44, but may alternatively comprise any other suitable number of field-generators 44. Pad 40 further comprises a pillow (not shown) placed under head 41 of patient 22, such that field-generators 44 are located at fixed and known positions external to head 41.

In some embodiments, system 20 comprises a console 33, which comprises a memory 49, and a driver circuit 42 configured to drive, via a cable 37, field-generators 44 with suitable signals so as to generate magnetic fields in a predefined working volume in space around head 41.

In some embodiments, console 33 comprises processor 34, typically a general-purpose computer, with suitable front end and interface circuits for receiving signals from ENT tool 28 having multiple magnetic sensors (not shown) coupled thereto, via a cable 32, and for controlling other components of system 20 described herein.

In some embodiments, processor 34 is configured to estimate the position of each position sensor. Based on the estimated positions of the respective sensors, in the coordinate system of the magnetic position tracking system, processor 34 is configured to derive the position, orientation and radius of curvature of a deflected distal end of ENT tool 28 that is shown in FIGS. 2A and 2B below.

In the context of the present invention and in the claims, the terms “bending” “deflecting” are used interchangeably and refer to deflection or bending of one or more sections of ENT tool 28 as will be described in detail in FIGS. 2A and 2B below.

In some embodiments, processor 34 is configured to receive via an interface (not shown), one or more anatomical images, such as computerized tomography (CT) images depicting respective segmented two-dimensional (2D) slices of head 41, obtained using an external CT system (not shown). The term “segmented” refers to displaying various types of tissues identified in each slice by measuring respective attenuation of the tissues in the CT system.

Console 33 further comprises input devices 39 for controlling the operation of system 20, and a user display 36, which is configured to display the data (e.g., images) received from processor 34 and/or to display inputs inserted by physician 24 or another user of input devices 39.

In some embodiments, processor 34 is configured to select one or mode slices from among the CT images, such as an anatomical image 35, and to display the selected slice on user display 36. In the example of FIG. 1, anatomical image 35 depicts a sectional front-view of one or more sinuses 48 of patient 22.

In some embodiments, processor 34 is configured to register between the coordinate systems of the CT system and the magnetic position tracking system, and to overlay the position of the distal end of ENT tool 28, on anatomical image 35.

FIG. 1 shows only elements related to the disclosed techniques, for the sake of simplicity and clarity. System 20 typically comprises additional modules and elements that are not directly related to the disclosed techniques, and therefore, are intentionally omitted from FIG. 1 and from the description of system 20.

Processor 34 may be programmed in software to carry out the functions that are used by the system, and to store data in memory 49 to be processed or otherwise used by the software. The software may be downloaded to the processor in electronic form, over a network, for example, or it may be provided on non-transitory tangible media, such as optical, magnetic or electronic memory media. Alternatively, some or all of the functions of processor 34 may be carried out by dedicated or programmable digital hardware components.

### Deflectable and Rotatable Distal-End Assembly Having Intrusions and Protrusions Patterned in a Single Tube

FIG. 2A is a schematic, pictorial illustration of ENT tool 28 having a deflectable and rotatable distal-end assembly 134 in a straight position, in accordance with an embodiment of the present invention. In some embodiments, shaft 38 and distal-end assembly 134 are configured to rotate about a longitudinal axis 50 of ENT tool 28. The rotation capability is represented by an arrow 43. Note that the rotation may be carried out clockwise and/or counterclockwise so as to improve the maneuverability of distal-end assembly 134. In such embodiments, physician 24 may rotate both shaft 38 and distal-end assembly 134 together, by rotating a control handle 128 described in detail below. In alternative embodiments, physician 24 may rotate shaft 38 and distal-end assembly 134 separately.

Reference is now made to an inset 80. In some embodiments, distal-end assembly 134 comprises a hollow tube 66, which is coupled to the distal end of shaft 38 and is typically made from a single piece of any suitable material, such as but not limited to a suitable alloy of nickel and titanium, e.g., Nitinol™ or super-elastic Nitinol™, having high repeatability.

In some embodiments, tube 66 is sized and shaped for being comfortably inserted through nose 26 into sinuses 48 or any other organ in head 41 of patient 22. Tube 66 is also sized and shaped for allowing a medical instrument, such as a sinuplasty balloon, a surgical tool, a suction or irrigation tool, or any other suitable tool, to be subsequently inserted through a lumen 140 of tube 66, which is described in detail below.

In some embodiments, tube 66 of distal-end assembly 134 has multiple slots, such as slots 77A, 77B, 77C and 77D, each of which formed at a respective section, also referred to herein as a rib, of distal-end assembly 134.

In the example of FIG. 2A, slot 77A is the distal-most patterned section of distal-end assembly 134 and has the largest size from among slots 77A-77D patterned in tube 66. Similarly, slot 77D is the proximal-most patterned section of distal-end assembly 134 and has the smallest size from among slots 77A-77D of tube 66. As will be depicted in FIG. 2B below, the slot size determines the bending limit of the respective section of distal-end assembly 134.

In some embodiments, each of slots 77A-77D has one or more protrusions and intrusions, which may be formed, by laser cutting or using any other suitable technique, on a section of the circumference of tube 66.

Reference is now made to an inset 92, showing slot 77A of tube 66. In some embodiments, slot 77A comprises multiple intrusions 99A, 99B, 99C, 99D and 99E, which are sized and shaped to fit snugly over respective protrusions 88A, 88B, 88C, 88D and 88E of tube 66. For example, intrusion 99A is adapted to fit over protrusion 88A, and intrusion 99D is adapted to fit over protrusion 88D.

Reference is now made back to inset 80. In some embodiments, when distal-end assembly 134 is in a straight position, also referred to herein as unflexed state (as shown in inset 80), protrusion 88A and intrusion 99A are may be disengaged from one another, whereas protrusion 88D and intrusion 99D are partially engaged with one another. In the straight position, slot 77A has the maximal size, e.g., along longitudinal axis 50. In other embodiments, distal-end assembly 134 may have any other suitable configuration. For example, in the straight position protrusion 88A and intrusion 99A may be at least partially engaged with one another. Moreover, in the straight position at least one protrusion may be partially or fully inserted into the respective intrusion.

Reference is now made back to inset 92. In some embodiments, protrusion 88A has surfaces 55A and 55B, and intrusion 99A has surfaces 56A and 56B. Note that when tube 66 is not deflected (e.g., in the unflexed state), surfaces 55A and 56A are not facing one another. Similarly, when tube 66 is not deflected, surfaces 55B and 56B are not facing one another.

In some embodiments, tube 66 has a non-patterned surface, referred to herein as a spine 78. In some embodiments, slots 77A-77D are patterned along the circumference of tube 66 and are therefore circular. The circular slots may be formed on any suitable portion of the circumference of tube 66. For example, at least one of the circular slots may cover between about 20% and about 95% of the circumference of tube 66.

In some embodiments, slots 77A-77D may be patterned symmetrically along the circumference of tube 66. For example, two sets of circular slots, such as slots 77A-77D, may be patterned symmetrically with the aforementioned protrusions and intrusions at both sides of spine 78.

In some embodiments, tube 66 may have an additional pattern connecting between the circular slots. In the example of FIG. 2A, the additional pattern is mechanically connecting between protrusions 88A (and intrusions 99A) of slots 77A that are extended from both sides of spine 78.

In the example configuration of FIG. 2A, tube 66 has ten slots, but in other configurations tube 66 may have any suitable number of slots, e.g., between 3 and 20, 4 and 20, 5 and 20, 6 and 20, 7 and 20, 8 and 20, 9 and 20, 10 and 20, 11 and 20, 12 and 20, 13 and 20, 14 and 20, and 15 and 20 slots having any suitable size and shape. Note that the slots may have a similar shape and different size, or a different shape, or any suitable combination of the above.

In alternative embodiments, the size of the slots may gradually increase from the proximal end to the distal end. In other embodiments, the size of the slots may alter along longitudinal axis 50. For example, slot 77A may be larger than slot 77B, but smaller than the size of slot 77C.

In yet other embodiments, the size of the slots, protrusions and intrusions of tube 66 may have any other suitable distribution along longitudinal axis 50. Additionally or alternatively, the size of the protrusions and intrusions of tube 66 may have any other suitable distribution across longitudinal axis 50.

Reference is now made back to the general view of FIG. 2A. In some embodiments, ENT tool 28 comprises control handle 128, which is coupled to handheld apparatus 30 shown in FIG. 1 above and is fitted at the proximal end of shaft 38. Control handle 128 is configured to bend and straighten distal-end assembly 134 relative to longitudinal axis 50 of ENT tool 28.

Reference is now made to an inset 70, which is a traversing sectional view BB of shaft 38. In some embodiments, shaft 38 is hollow and shaped to define a tube lumen 140. ENT tool 28 comprises a pull wire 130, made from or comprising a suitable alloy of nickel and titanium, such as Nitinol™ or other suitable materials, which passes proximally-distally through tube lumen 140.

In some embodiments, pull wire 130 may be connected to a ring or any other element coupled to a selected section, such as the distalmost section, of hollow tube 66, for example, distal to slot 77A. As will be described in FIG. 2B below, pull wire 130 facilitates adjusting the configuration of the distal portion of tube 66. In other embodiments, ENT tool 28 may comprise a ribbon (not shown), instead of, or in addition to pull wire 130. The ribbon may comprise Nitinol™ or any other suitable material. A configuration of the aforementioned ribbon in an ENT tool is described in detail in U.S. Patents Application Publications 2017/0325841, which is incorporated herein by reference.

As shown in FIG. 2A, in the unflexed state of tube 66, each of the slots, and sections between the slots, is generally flush with its neighbors along longitudinal axis 50 at the circumference of tube 66.

Reference is now made to an inset 60, which is a sectional view AA of control handle 128 along longitudinal axis 50. As described above, control handle 128 is fitted at the proximal end of shaft 38. In some embodiments, control handle 128 is rotatable and is configured to control pulling wire 130.

As will be described in FIG. 2B below, by turning control handle 128 in one direction, pull wire 130 is pulled, thus causing flexion of distal-end assembly 134. Conversely, by turning control handle 128 in the opposite direction, pull wire 130 is pushed, thus causing tube 66 of distal-end assembly 134 to be unflexed and straight as shown in FIG. 2A.

For example, the proximal end of pull wire 130 may be coupled to a sliding element 142, which is configured to slide between an extreme proximal position and an extreme distal position. By turning control handle 128, sliding element 142 is slid proximally or distally, thus causing tube 66 to be flexed or unflexed. In some embodiments, the inner surface of control handle 128 may be shaped to form a female threading 144, and control handle 128 may also comprise a complementary male threading 146, which is engaged with female threading 144. In an embodiment, when physician 24 (or any other operator of ENT tool 28) turns control handle 130, male threading 146, which is coupled to sliding element 142, moves the sliding element proximally or distally along longitudinal axis 50, and controls the state of tube 66 as described above.

In some embodiments, the protrusion and intrusions of distal-end assembly 134 (e.g., protrusions 88 and intrusions 99) may have any suitable shape, such as but not limited to, rectangular, parallelogram, trapezoid, dome shaped, pyramid shape, or any type of polygon.

This particular configuration of distal-end assembly 134 is shown by way of example, in order to illustrate certain problems that are addressed by embodiments of the present invention and to demonstrate the application of these embodiments in enhancing the performance of such a medical probe (e.g., ENT tool 28). Embodiments of the present invention, however, are by no means limited to this specific sort of example configuration of ENT module, and the principles described herein may similarly be applied to other sorts of medical probes.

FIG. 2B is a schematic, pictorial illustration of ENT tool 28 having distal-end assembly 134 in a deflected position, in accordance with an embodiment of the present invention.

Reference is now made to an inset 90 showing a longitudinal cross-section of control handle 128. As described in FIG. 2A above, when physician 24 turns control handle 128, pull wire 130 is pulled along longitudinal axis 50 towards the proximal end of ENT tool 28, and tube 66 is deflected. In some embodiments, control handle 128 is further configured, subsequently to the aforementioned turning, to hold the ribbon in place, thus to maintain the position of tube 66. For example, the engagement of threading 144 with threading 146, as shown in FIG. 2B, may prevent the sliding element from sliding.

In other embodiments, control handle 128 may comprise any other suitable mechanism for preventing undesired sliding of the aforementioned pull wire or ribbon or any other mechanism suitable for deflecting distal-end assembly 134 of ENT tool 28.

Reference is now made to an inset 100 showing distal-end assembly 134 in a fully deflected position. In some embodiments, when pull wire 130 is pulled proximally along longitudinal axis 50, tube 66 is deflecting and the protrusions of tube 66 are inserted into the respective intrusions thereof.

Reference is now made to an inset 150 showing the protrusions and intrusions of slot 77A. In some embodiments, in the fully deflected position shown in FIG. 2B, protrusions 88A, 88B, 88C, 88D and 88E of slot 77A, are inserted into intrusions 99A, 99B, 99C, 99D and 99E, respectively. In such embodiments, surface 55A of protrusion 88A and surface 56A of intrusion 99A are facing one another and are typically in physical contact with one another. Similarly, surface 55B of protrusion 88A and surface 56B of intrusion 99A are facing one another and are typically in physical contact with one another.

In some embodiments, when tube 66 is deflected and rotated at the same time (as shown in the general view of FIG. 2B), surfaces 55A and 56A apply force to one another and thus resist the rotation of tube 66. Similarly, when tube 66 is deflected and rotated at the same time, surfaces 55B and 56B apply force to one another. Note that the size and shape of the protrusions and intrusions of tube 66 allow complete insertion of the protrusions into the respective intrusions, and physical contact, without sliding, between the aforementioned surfaces.

In some embodiments, the size of the outer and inner diameters of tube 66 may be about 4.2 mm and 3.6 mm, respectively. Therefore, the wall thickness of tube 66 (i.e., between the outer and inner surfaces) may be about 0.2 mm, and the size tolerance of the aforementioned protrusions and intrusions may be about 0.01 mm.

In other embodiments, the inner and outer diameter, and the wall thickness may have any other size suitable for a respective medical procedure carried out in a respective organ of patient 22.

In the context of the present disclosure and in the claims, the terms “about” or “approximately” for any numerical values or ranges indicate a suitable dimensional tolerance that allows the part or collection of components to function for its intended purpose as described herein. For example, “about” or “approximately” may refer to the range of values ±20% of the recited value, e.g. “about 90%” may refer to the range of values from 72% to 100%.

In such embodiments, when tube 66 is deflected and rotated at the same time, surfaces 55A and 56A, and surfaces 55B and 56B, may not slide relative to one another. Rather, surfaces 55A and 56A may press one another, or may apply any other force (e.g., a combination of pressing and shearing and/or friction) to one another. When tube 66 is deflected and, at the same time, is also rotated against a bone or another rigid tissue, the inter-surface force described above improves the resistance of tube 66 to rotation, and therefore improves the durability of tube 66 against breakage. The inventors found that, compared to a deflectable Nitinol™ tube having fewer pairs of protrusions and intrusions with a length of about 40 mm and a wall thickness of 0.2 mm, the breakage resistance of tube 66 having the same material, length and wall thickness, may increase about fourfold by having slots 77, protrusions 88 and intrusions 99 with the tolerances described above. An example configuration of fewer pairs of protrusions and intrusions is shown, for example, in U.S. patent application Ser. No. 16/421,430 filed May 23, 2019, which is assigned to the assignee of the present patent application and whose disclosure is incorporated herein by reference.

As described in FIG. 2A above, each of slots 77A-77D has a slit, formed on a section of the circumference of tube 66. The size (e.g., width) of the slit determines the slot angle when physician 24 is bending tube 66. In the example of FIG. 2B, the slit of slot 77A is larger than the slit of slot 77C, and therefore, the slot angle of slot 77A is larger than the slot angle of slot 77C. Note that in the unflexed state shown in FIG. 2A above, physician 24 is not bending tube 66 and therefore the sections of tube 66 remain flush with one another.

In some embodiments, the slit size and/or the size of the protrusion and intrusion typically determine the maximal amount of deflection of the respective section. The maximal deflection ability at a given location along tube 66 may be specified by a local radius of curvature (LROC) at that location.

In such embodiments, a larger slot, protrusion and intrusion enable increased the amount of deflection, measured by a smaller LROC. As shown in inset 100, R_{A}, which is the LROC of slot 77A is smaller than R_{C}, which is the LROC of slot 77C. Note that the term “local” refers to the arc formed by the bending of the outer surface of tube 66 at the position of the respective slot.

This configuration refers to the sizes of the remaining slots and respective LROCs of tube 66 are within the ranges defined above between slots 77A and 77D. For example, at maximal deflection, the LROC of slot 77C is typically smaller than that of slot 77D, and is larger than that of slot 77A. Note that the dimensions described above are provided by way of example, and in other embodiments, the slots, intrusions, protrusions and LROCs may have any other suitable dimensions.

Note that the LROCs described above are indicative of the LROC at a maximal deflection or bending of tube 66 at each respective section. In some embodiments, physician 24 may apply less-than maximal bending to tube 66 by applying a smaller turning or rotation angle to control handle 128. In such embodiments, only a portion of a given protrusion (e.g., protrusion 88A) may be inserted into the respective intrusion (e.g., intrusion 99A), and the LROC may be larger compared to the fully deflected LROC shown in inset 100.

In some embodiments, when physician 24 moves ENT tool 28 in head 41 of patient 22, at least one section of tube 66 may by fully deflected and another section may be partially deflected or not deflected at all. For example, physician 24 may position distal-end assembly 134 at the ostium of sinus 48 (as shown in FIG. 1 above), and subsequently deflect only the third distalmost part of tube 66.

In this example embodiment, the ostium of sinus 48 may fix the sections of slots 77C and 77D to be substantially flush with shaft 38 (as shown in FIG. 2A above), physician 24 may partially deflect the section comprising slot 77B, whereas the section comprising slots 77A may be fully deflected to obtain the R_{A} LROC shown in inset 100. In other example embodiments, physician 24 may partially or fully deflect any other one or more sections of tube 66 so as to maneuver distal-end assembly 134 to a desired location in head 41, e.g., based on the tracked position of distal-end assembly 134 in anatomical image 35 shown in FIG. 1 above.

Although the embodiments described herein mainly address medical probes used minimally invasive procedures carried out in the ear-nose-throat (ENT) of a patient, the methods and systems described herein can also be used in other applications.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical probe, comprising:
a shaft for insertion into a cavity of a patient body; and
a distal-end assembly, coupled to a distal end of the shaft and comprising a hollow tube, which is configured to deflect relative to a longitudinal axis of the hollow tube and to rotate about the longitudinal axis, the hollow tube having (i) an intrusion, having at least a first surface, and (ii) a protrusion, which is facing the intrusion and having a second surface,
wherein, when the hollow tube is deflected, at least part of the protrusion protrudes into the intrusion so that a first section of the first surface and a second section of the second surface are facing one another, and when the hollow tube is deflected and rotated, the first and second sections of the first and second surfaces apply force to one another and thus resist rotation of the hollow tube.

2. The medical probe according to claim 1, wherein the distal-end assembly comprises (i) a first slot, located at a first section along the longitudinal axis of the hollow tube, and having a first size that limits bending of the first section by a first local radius of curvature (LROC), and (ii) a second slot, located at a second different section along the longitudinal axis of the hollow tube, and having a second different size that limits bending of the second section by a second different LROC.

3. The medical probe according to claim 2, wherein at least the first slot comprises (i) a plurality of the intrusions having respective one or more first surfaces, and (ii) a plurality of the protrusions having respective one or more second surfaces, and wherein the intrusions and protrusions are arranged along at least the first slot.

4. The medical probe according to claim 3, wherein at least the first slot comprises at least a given intrusion having a first given surface, and a given protrusion, which is facing the given intrusion and having a second given surface, wherein when the hollow tube is not deflected, the first and second given surfaces do not apply force to one another.

5. The medical probe according to claim 2, and comprising a control handle, fitted at a proximal end of the shaft and configured to bend the first section by up to the first LROC and the second section by up to the second LROC.

6. The medical probe according to claim 1, wherein the distal-end assembly comprises an alloy of nickel and titanium.

7. The medical probe according to claim 1, wherein the intrusion is shaped to fit over the protrusion, such that, when the hollow tube is deflected and rotated, the first and second sections do not slide relative to one another.

8. The medical probe according to claim 1, wherein the first and second sections of the first and second surfaces press against one another.

9. The medical probe according to claim 1, wherein the protrusion has a shape selected from a list of shapes consisting of: rectangular, parallelogram, trapezoid, dome, and pyramid.

10. The medical probe according to claim 1, wherein the cavity comprises an ear-nose-throat (ENT) sinus.

11. A method for producing a medical probe, the method comprising:
providing a shaft for insertion into a cavity of a patient body; and
coupling, to a distal end of the shaft, a distal-end assembly comprising a hollow tube that deflects relative to a longitudinal axis of the hollow tube and rotates about the longitudinal axis, the hollow tube having (i) an intrusion, having at least a first surface, and (ii) a protrusion, which is facing the intrusion and having a second surface,
wherein when the hollow tube is deflected, at least part of the protrusion protrudes into the intrusion so that a first section of the first surface and a second section of the second surface are facing one another, and when the hollow tube is deflected and rotated, the first and second sections of the first and second surfaces apply force to one another and thus resist rotation of the hollow tube.

12. The method according to claim 11, wherein the distal-end assembly comprises (i) a first slot, located at a first section along the longitudinal axis of the hollow tube, and having a first size that limits bending of the first section by a first local radius of curvature (LROC), and (ii) a second slot, located at a second different section along the longitudinal axis of the hollow tube, and having a second different size that limits bending of the second section by a second different LROC.

13. The method according to claim 12, wherein at least the first slot comprises (i) a plurality of the intrusions having respective one or more first surfaces, and (ii) a plurality of the protrusions having respective one or more second surfaces, and wherein the intrusions and protrusions are arranged along at least the first slot.

14. The method according to claim 13, wherein at least the first slot comprises at least a given intrusion having a first given surface, and a given protrusion, which is facing the given intrusion and having a second given surface, wherein when the hollow tube is not deflected, the first and second given surfaces do not apply force to one another.

15. The method according to claim 12, and comprising fitting, at a proximal end of the shaft, a control handle for bending the first section by up to the first LROC and the second section by up to the second LROC.

16. The method according to claim 12, and comprising forming at least one of the first and second slot using a laser cutting technique.

17. The method according to claim 11, wherein the intrusion is shaped to fit over the protrusion, such that, when the hollow tube is deflected and rotated, the first and second sections do not slide relative to one another.

18. The method according to claim 11, wherein the first and second sections of the first and second surfaces press against one another.

19. The method according to claim 11, wherein the protrusion has a shape selected from a list of shapes consisting of: rectangular, parallelogram, trapezoid, dome, and pyramid.

20. The method according to claim 11, wherein the distal-end assembly comprises an alloy of nickel and titanium.
